(19) United States
(12) Patent Application Publication
(10) Pub. No.: US 2004/0028702 A1
(43) Pub. Date: Feb. 12, 2004

(54) Muramic acid derivative compounds

(76) Inventors: Milana M. Maletic, Summit, NJ (US); Aaron H. Leeman, Westfield, NJ (US); Gina M. Santorelli, Oceanport, NJ (US); Sherman T. Waddell, Westfield, NJ (US)

Correspondence Address: Mollie M. Yang, Merck & Co., Inc., Patent Dept., RY60-30, P. O. Box 2000, Rahway, NJ, 07065-0907, US

(21) Appl. No.: 10/616,189

(22) Filed: Jul. 9, 2003

Related U.S. Application Data
(60) Provisional application No. 60/398,981, filed on Jul. 26, 2002.

(51) Int. Cl. A61K 39/02; C08B 37/00; A61K 31/739

(52) U.S. Cl. 424/234.1; 514/54; 536/53

(57) ABSTRACT

The present invention pertains to the combinatorial synthesis of antibacterial agents that inhibit the Mur-pathway enzymes involved in bacterial cell wall assembly. The method uses p-alkoxybenzylidene as the linker for the attachment of a derivatized muramic acid to the polymeric resin support. This intermediate becomes the starting point for the combinatorial synthesis, which in conjunction with split-pooling techniques allows rapid synthesis of diverse analogues for high-throughput screening (HTS). The invention is further directed to the libraries of these finished compounds that vary at the peptide, N-acyl and anomeric UDP positions.

### CROSS-REFERENCE TO RELATED APPLICATION

This application claims the benefit of U.S. Provisional Application No. 60/398,981, filed Jul. 26, 2002.

### FIELD OF THE INVENTION

This invention relates to muramic acid derivative compounds and a method for the combinatorial synthesis and identification of said compounds as potential inhibitors of enzymes involved in cell wall biosynthesis. This invention also relates to a method for the preparation of intermediates of muramic acid linked to polymer beads by an acetal linker and its subsequent use in the synthesis of solid phase libraries of compounds to be screened as antibacterial agents.

### BACKGROUND OF THE INVENTION

Bacterial infections remain among the most common and deadly causes of human disease. Unfortunately, the overuse of antibiotics has led to antibiotic-resistant pathogenic strains of bacteria. Indeed, bacterial resistance to the new chemical analogues of these drugs appears to be out-pacing the development of new analogues. For example, life-threatening strains of three species of bacteria (Enterococcus faecalis, Mycobacterium tuberculosis, and Pseudomonas aeruginosa) have evolved to be resistant against all known antibiotics. [Stuart B. Levy, “The Challenge of Antibiotic Resistance”, in Scientific American, pg. 46-53 (March 1998)]. New approaches to drug development are therefore necessary to combat the ever-increasing number of antibiotic-resistant pathogens.

The molecular target of current antibiotics, including natural products (bacitracin, fosfomycin, and D-cycloserine), glycopeptides (vancomycin) and β-lactams (penicillin and methicillin), is the synthesis of the bacterial cell wall. The cell wall consisting of glycopeptide polymeric murein (peptidoglycan) completely encloses and stabilizes the bacterial cell [Weidel & Pelzer et al., Enzymol, 26:193-232 (1964)]. The mechanism by which these antibiotics attack reveals the significance of cell wall biosynthesis as a particularly effective point of attack against bacteria.

The cell wall, which defines the boundary and shape of the bacterial cell, is composed of the polymeric peptidoglycan matrix consisting of alternating N-acetylmuramic acid (MurNAc) and N-acetylglucosamine (GIcNAc) units. These assembled glycan chains are cross-linked with pentapeptide bridges thereby providing the strength the structure requires to maintain cell integrity against osmotic pressure differential of over four atmospheres, yet provide the flexibility for the cell to move, grow and divide. (Rogers, H. J., H. R. Perkins, and J. B. Ward, 1980, Biosynthesis of Peptidoglycan, pp. 239-297; Chapman & Hall Ltd. London).

The monomeric precursor for peptidoglycan biosynthesis is assembled in the cytoplasm starting with the condensation of an activated sugar molecule UDP-N-acetylglucosamine with phosphoenolpyruvate (PEP) as catalyzed by the enzyme MurA. Carboxylate, generated by reduction with NADPH and enzyme MurB, serves as the point of attachment for the pentapeptide chain, which is in turn synthesized by the other Mur-pathway enzymes, specifically a series of ATP-dependent amino acid ligases (MurC, MurD, MurE and MurF). The placement of a lactyl group on the 3-OH of the glucosamine moiety by MurB generates lactyl carboxylate as the first acceptor site. After attachment of the sugar pentapeptide to a lipid carrier in the plasma membrane, another glucosamine unit is added to the 4-OH of the muramic acid moiety. The completed monomeric building block is then moved across the membrane into the periplasm where the penicillin-binding proteins enzymatically add it into the growing cell wall.

Disruption of the murein structure leads to cell lysis; thus, supporting that peptidoglycan biosynthesis is an essential pathway and an important potential target for antibiotic research. As antibacterial targets, Mur-pathway enzymes have not been thoroughly examined for inhibition because the unavailability of substrates. Although a high-throughput coupled enzyme assay that is capable of simultaneously screening for inhibitors of any one of the Mur-pathway enzymes is available [Wong, et al. J. Am. Chem. Soc., 120 pp. 13527-13528 (1998)], the challenge remains in the development of an efficient synthesis of inhibitors for these enzymes.

An efficient screening method, in conjunction with rapid analogue synthesis of Mur-pathway enzyme inhibitors, would accelerate the discovery of the next generation of antibiotics. In the past decade solid phase combinatorial chemistry has emerged as a very powerful tool for the discovery of biologically active compounds and for rapid optimization of lead structures as pharmaceutical agents. The “split-pool technique” involves dividing a reaction mixture containing the core structure into separate reaction vessels where each will be subject to different additional synthesis steps to create diverse compounds. This allows for rapid synthesis of large libraries of compounds, but assaying mixtures may give false results and require labor-intensive deconvolution. Parallel synthesis of single compounds avoids these screening problems, but is limited to the synthesis of smaller libraries. Since each bead in the split-pool combinatorial synthesis undergoes a set of unique chemical reactions, at the end of the sequence it carries only a single chemical entity. Creating the solid phase library using the split-pool technique, but separating the beads prior to cleavage and assay, retains the synthetic advantages of split-pool technique while also having the analytical advantages of parallel synthesis.

The increased prevalence and rapid emergence of drug resistance to the current family of antibiotic agents demand new approaches to antibiotic development. Chemical disruption of the Mur-pathway enzymes stands as a valid target for the halt of cell wall construction. The chemical diversity of compounds being generated using combinatorial chemistry coupled with rapid screening programs offers the opportunity to identify new classes of antibacterial agents. Accordingly, the development of new drugs, which affect an alternative bacterial target protein, would be desirable.

### SUMMARY OF THE INVENTION

The present invention pertains to the combinatorial synthesis of antibacterial agents that inhibit the Mur-pathway enzymes involved in bacterial cell wall assembly. The method uses p-alkoxybenzylidene as the linker for the attachment of a monosaccharide, specifically a derivatized muramic acid, to the polymeric resin support. This intermediate becomes the starting point for the combinatorial synthesis, which in conjunction with split-pooling techniques allows rapid synthesis of diverse analogues for high-throughput screening.

The present invention is further directed to muramic acid derivative compounds, which are inhibitors of the Mur pathway enzymes, their use as antibacterial agents, and pharmaceutical compositions containing them.

The present invention is further directed to the libraries of muramic acid derivatives that vary at the peptide, N-acyl and anomeric UDP positions. The diversity of compounds allows easier and rapid identification of potential therapeutics and antibacterial agents with the use of HTS screening.

### DETAILED DESCRIPTION OF THE INVENTION

The present invention provides compounds of Formula Ia:

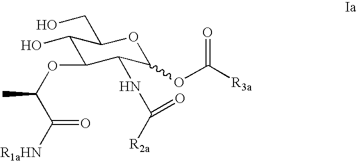

or a pharmaceutically acceptable salt thereof wherein

R₁ₐ is selected from:

1) —C₁₋₁₀alkyl-CO₂R^{a},

2) —CH(CO₂R^{a})CH₂CH₂CO₂R^{b}, and

3) —CH(CO₂R^{a})CH₂OR^{b};

R₂ₐ is selected from:

1) C₁₋₁₀alkyl,

2) —NR^{a}(C₁₋₁₀alkyl),

3) —CH₂OR^{a},

4) C₃₋₆cycloalkyl,

5) Ar, and

6) —N(R^{a})—Ar;

R₃ₐ is selected from:

1) —N(R^{a})—Ar,

2) —CH═CH₂—Ar,

3) —NHSO₂—Ar, and

4) —(CH₂)₂₋₅—C(O)—3-thienyl;

Ar is phenyl optionally substituted with 1 to 2 groups independently selected from halogen and C₁₋₄alkyl; and

R^{a} and R^{b} are independently selected from hydrogen and C₁₋₁₀ alkyl.

In one embodiment of Formula Ia are compounds wherein R₁ₐ is —CH(CO₂H)CH₂CH₂CO₂H.

In a second embodiment of Formula Ia are compounds wherein R₂ₐ is C₁₋₆alkyl.

In a third embodiment of Formula Ia are compounds wherein R₃ₐ is —NHSO₂—Ar.

Another aspect of the present invention provides a method for solid phase combinatorial synthesis of libraries of potential Mur enzyme inhibitors of formula I for high throughput screening. It has been shown that the MurNAc moiety is an important contributor to the potency of some Mur D enzyme inhibitors, since it displays the UDP, N-acyl, and pentapeptide moieties around a rigid scaffold in a predictable orientation. Therefore, in designing prototype libraries of MurNAc derivatives, the sugar is left unaltered in the scaffold, but the other sites—the peptide position, the N-acyl position and the polar anomeric UDP position—are modified. As a result, the ideal point for anchoring the sugar to solid phase is the 4,6-diol unit of MurNAc.

In the present method, MurNAc or a derivative thereof is first attached via the 4,6-diol group to the solid phase through a p-alkoxybenzylidene linker derived from 4-(formyl)phenoxyacetic acid acetal. The bound MurNAc derivative is then subject to further chemical manipulation to create libraries of compounds of formula I. In one embodiment for attaching MurNAc or a derivative thereof, as depicted in Scheme 1, a suitably protected MurNAc derivative [1] is first coupled with a suitably protected 4-(formyl)phenoxyacetic acid acetal [2] in the presence of an acid, such as p-toluenesulfonic acid, to provide the diprotected 4,6-O-benzylidene-MurNAc derivative intermediate [3]. Following the protection of the anomeric hydroxy group and the unmasking of the phenoxyacetic acid, intermediate [3] is attached to the solid phase having a free amino group using conventional amide forming chemistry to provide solid phase bound MurNAc derivative [4]. The selection of the various protecting groups, as well as methods for introducing and removing such groups are within the knowledge of one skilled in the art of organic synthesis. Examples of suitable protecting group are trimethylsilylethyl for the carboxy group of MurNAc, 9-fluorenylmethyl for the carboxy group of 4-(formyl)phenoxyacetic acid, and allyloxy-carbonyl for the anomeric hydroxy group of MurNAc.

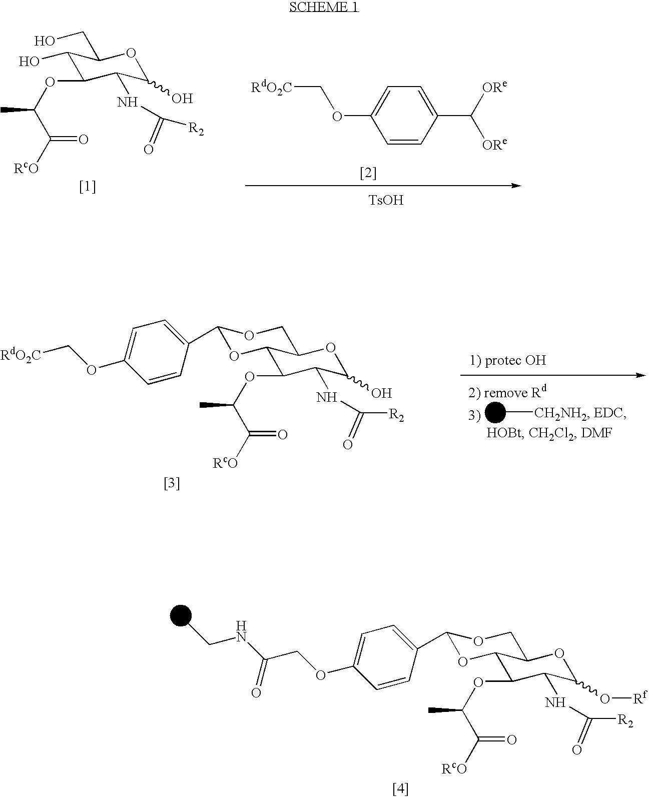

R^{c} and R^{d} are different carboxy protecting groups; R^{e} is C₁₋₃alkyl; and R^{f} is a hydroxy protecting group.

In an alternate embodiment for attaching MurNAc or a derivative thereof to the solid support, as depicted in Scheme 2, the linker 4-(formyl)phenoxy-acetic acid is first attached to the solid phase via an amide bond to form the resin bound linker [5], and the aldehyde group is then activated as an acetal to form [6]. Coupling the bound acetal [13] with a suitably protected MurNAc derivative [1] and, protection of the anomeric hydroxy group provides the bound MurNAc derivative [4]. MurNAc derivative is directly attached onto the activated resin by reacting only two equivalents of sugar with respect to the reactive sites on the polymeric resin support to accomplish high loading.

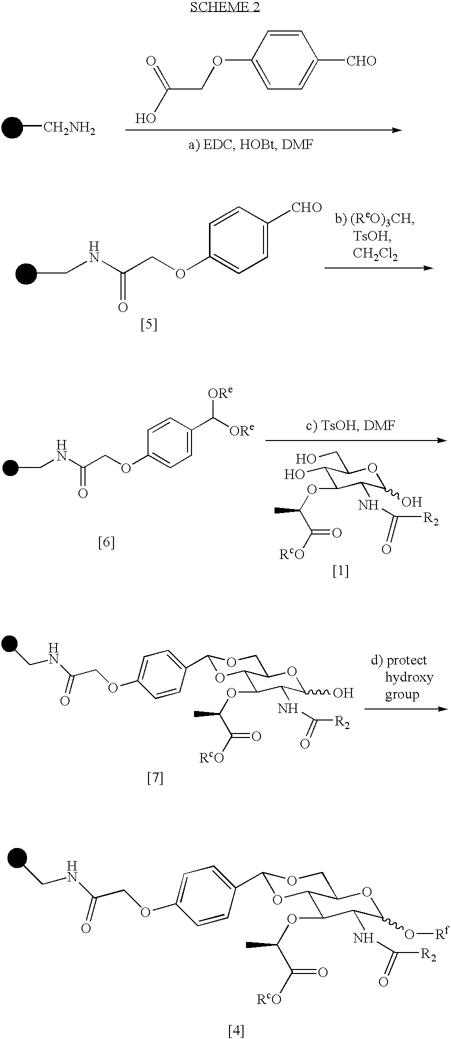

R^{c} is carboxyl protecting group; R^{e} is C₁₋₃alkyl; and R^{f} is hydroxy protecting group

Using a split-pool technique the solid phase bound MurNAc derivative [4] is elaborated at the anomeric hydroxy group and at the carboxy group as depicted in Scheme 3 to provide libraries of potential Mur-pathway inhibitors. The carboxylate of the resin bound MurNAc derivative is unmasked, and the resulting resin split into separate vessels. The resin in each vessel is coupled with a different protected amino compound to give the corresponding amides [8]. These amides are mixed and the anomeric position is deprotected. The resin is again split into separate vessels, and the content of each is acylated to provide anomeric O-acyl derivatives [9]. After pooling the resins, the compounds are released from the resin by treatment with, for example, trifluoroacetic acid in methylene chloride at room temperature, or glacial acetic acid at 40° C. to give a library of compounds of formula I.

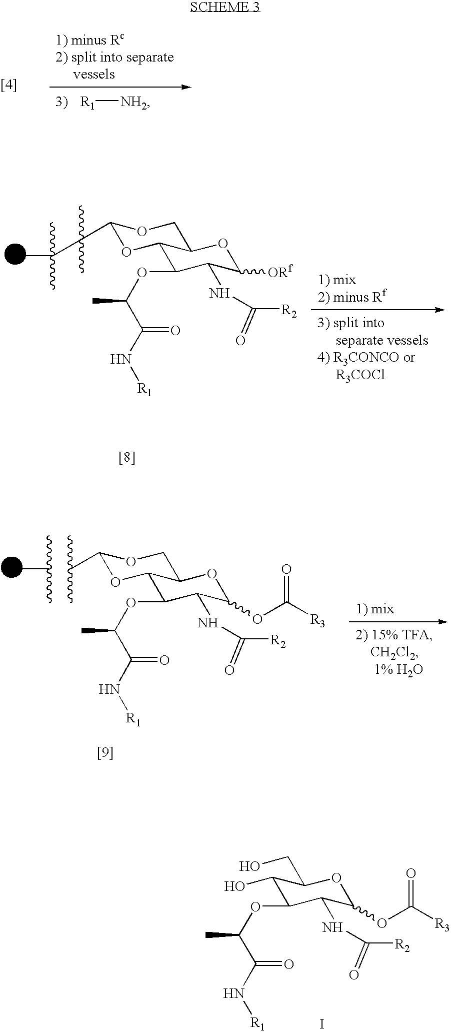

The method described in Schemes 1-3 may be used to create libraries of compounds of formula I that vary at one or more of R₁, R₂ and R₃ sites. R₁, R₂ and R₃ may be an organic radical as the method can accommodate wide variation of R₁, R₂ and R₃. The identity of each group is not critical so long as it does not interfere with the chemical manipulations described in the schemes. For example, R₁ may be alkyl, alkenyl, alkynyl, aryl, heteroaryl, (hetero)aralkyl, (hetero)aralkenyl and cycloalkyl, each of which is optionally substituted; R₂ may be a group selected from R₁, NHR₁, NR₁R₁, OR₁, and NHSO₂R₁; and R₃ may be a group selected from R₁, NHR₁, NR₁R₁ and NHSO₂R₁. Libraries thus produced are screened using assays known in the art such as the high throughput assay described in Wong et al, J. Am. Soc. Chem., 1998, 120, 13527-13528, which is hereby incorporated by reference.

Compound [1], where R₂ is other than methyl, may be prepared from 2-acetamido-2-deoxyglucopyranoside derivative as depicted in Scheme 4. The benzyl protected compound [10] is deacetylated using potassium hydroxide in ethanol at elevated temperature, and then acylated with the appropriate acylating agent such as an acid chloride to give N-acyl derivative [11]. Treatment of [11] with S-(−)-2-chloropropionic acid, followed by protecting group manipulation provides N-acylmuramic acid derivatives [1].

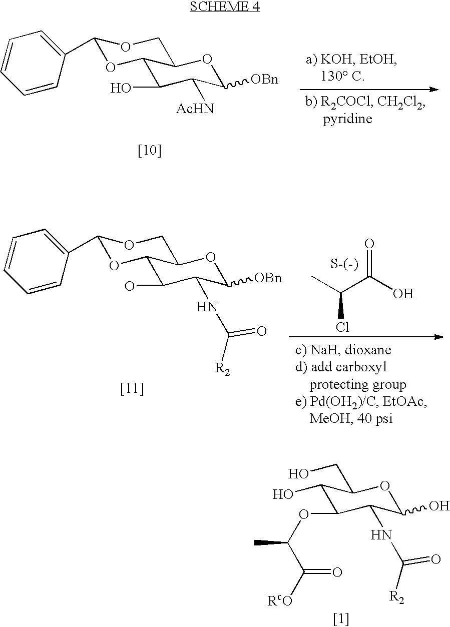

As used herein, unless indicated otherwise, the term “muramic acid derivative compound” means a compound that has muramic acid as its key monosaccharide and is chemically altered or protected to carry out additional synthetic chemistry for substituent modifications.

The term “library of compounds” or “combinatorial library” means a collection of compounds based upon a core structure, for example, muramic acid in the present invention, wherein the library contains a discrete number of independently variable substituents, functional groups or structural elements, and further, wherein the library is designed so that, for the range of chemical moieties selected from each of the independently variable substituents, compounds containing all possible permutations of those substituents will be present in the library. Thus, by way of illustration, if a core structure, labeled M, contains three independently variable substituents, labeled R₁, R₂, and R₃, and if R₁ is taken from m different chemical moieties, R₂ from n different chemical moieties and R₃ from p different chemical moieties (wherein m, n, and p are integers which define the size of the library, and which range between 1 to 1000; preferably between 1 to 100; most preferably between 1 to 20), then the library would contain m times n times p (m×n×p) different chemical compounds and all possible combinations of R₁, R₂, and R₃ would be present on the core structure M within that library. The methods for preparing combinatorial libraries of compounds are such that the molecularly diverse compound members of the libraries are synthesized simultaneously.

The terms “resin-bound synthesis” or “solid phase synthesis” are used herein interchangeably to mean one or a series of chemical reactions used to prepare either a single compound or a library of molecularly diverse compounds, wherein the chemical reactions are performed on a compound, suitably, a monosaccharide, specifically a muramic acid, which is bound to a polymeric resin support through an appropriate linkage, suitably a p-alkoxybenzylidene acetal linker.

The terms “resin,” “inert resin,” “polymeric resin,” or “polymeric resin support” are used herein at all occurrences to mean a bead or other solid support such as beads, pellets, disks, capillaries, hollow fibers, needles, solid fibers, cellulose beads, pore-glass beads, silica gels, grafted co-poly beads, polyacrylamide beads, latex beads, dimethylacrylamide beads optionally cross-linked with N,N′-bis-acryloyl ethylene diamine, glass particles coated with a hydrophobic polymer, etc. i.e. a material having a rigid or semi-rigid surface. The solid support is suitably made of, for example, cross linked polystyrene resin, polyethylene glycol-polystyrene resin, benzyl ester resins or benzhydrylamine resins and any other substance which may be used as such and which would be known or obvious to one of ordinary skill in the art.

The term “alkyl” is used herein at all occurrences to mean a straight or branched chain radical of 1 to 20 carbon atoms, unless the chain length is limited thereto, including, but not limited to methyl, ethyl, n-propyl, isopropyl, n-butyl, sec-butyl, isobutyl, tert-butyl, and the like. Preferably the alkyl chain is 1 to 10 carbon atoms in length, more preferably 1 to 8 carbon atoms in length.

The term “aryl” is used herein at all occurrences to mean 5-14 membered optionally substituted aromatic ring(s) or ring systems which may include bi- or tri-cyclic systems and one or more heteroatoms, wherein the heteroatoms are selected from oxygen, nitrogen or sulfur. Representative examples include, but are not limited to phenyl, naphthyl, pyridyl, quinolinyl, thiazinyl, isoquinoline, imidazole, furanyl, and the like.

The term “assay” is used herein at all occurrences to mean a binding assay or a functional assay known or obvious to one of ordinary skill in the art, including, but not limited to, the assays disclosed herein. A particular suitable assay for use according to the invention is disclosed by Wong, et al. J. Am. Chem. Soc., 120 pp. 13527-13528 (1998).

The compounds of formula Ia are inhibitors of Mur-pathway enzymes and therefore have application as antibacterial agents. In one aspect, the compounds are active against various Gram-positive and to a lesser extent Gram-negative bacteria, and accordingly find utility in human and veterinary medicine. In another aspect, the compounds are useful in screening for the presence of other compounds that are inhibitors of the enzymes in the Mur pathway.

The compound of formula Ia may be used in a variety of pharmaceutical preparations. Compositions for injection may be prepared in unit dosage form in ampoules or in multi-dose containers. The compositions may take such forms as suspensions, solutions, or emulsions, oily or aqueous in nature, and may contain various formulating agents, such as diluents, buffers, preservatives and the like. Hence, the compound is present in combination with these pharmaceutically acceptable carriers. Alternatively, the active ingredient may be in the form of a powder, which can be reconstituted with a suitable carrier such as sterile water, normal saline and the like at the time of administration. The powder can be in lyophilized or non-lyophilized form.

Oral compositions are typically in the form of tablets, capsules, solution or suspension. Such compositions may likewise be packaged in unit dose or multi-dose containers. In these oral compositions, the pharmaceutically acceptable carriers may be comprised of diluents, tabletting and granulating aids, lubricants, disintegrants, buffers, sweeteners, preservatives and the like.

Topical applications may be formulated with a pharmaceutically acceptable carrier in the form of hydrophobic or hydrophilic ointments, creams lotions, solution, paints, or powders.

The dosage to be administered depends to a large extent upon the condition and size of the subject being treated as well as the route and frequency of administration. The parenteral route (by injection) is preferred for generalized infection. Such matter, however, are typically left to the discretion of the clinician according to principles of treatment well known in the antibacterial arts.

Compositions for human delivery per unit dosage, whether liquid or solid, may contain from about 0.01% to about 99% active material, the preferred range being from about 10%-60%. The composition will generally contain from about 15 mg to 2000 mg of the active ingredient; however, in general, it is preferable to employ a dosage amount in the range of from about 250 mg to 1000 mg. In parenteral administrations, the unit dosage is usually the compound of Formula Ia in a sterile water or saline solution or in the form of a soluble powder intended for solution.

The preferred method of administration is parenterally by intravenous (i.v.) infusion. Alternatively the compound may be administered intramuscularly (i.m.).

For adults, a dose of about 5 to about 50 mg of Formula Ia antibacterial compound per kg of body weight is administered from 1 to 6 times per day. The preferred dosage ranges from about 250 mg to about 1000 mg of the compound given one to four times per day.

More specifically, for mild infections a dose of 250 mg two to four times daily is preferred. For moderate infections against highly susceptible Gram-positive organisms, a dose of 500 mg b.i.d. to q.i.d. is preferred. For severe, life-threatening infections against organisms at the upper limits of sensitivity to the antibiotic, a dose of about 1000-2000 mg two to six times daily is preferred.

For children, a dose of 5 to 25 mg/kg of body weight given 1 to 4 times per day is preferred; a dose of 10 mg/kg b.i.d, t.i.d. or q.i.d. is recommended.

The following examples are provided for the purpose of further illustration only and are not intended to be limitations on the disclosed invention.

### EXAMPLE 1

### Synthesis of Resin-Bound N-Acetylmuramic Acid—Direct Attachment of Linker to MurNAc

To synthesize a resin-bound N-acetylmuramic acid (MurNAc [4a]) as depicted in Scheme 1a, a commercially available N-acetyl-1-O-benzyl-4,6-O-benzylidene muramic acid was initially protected as a 2-(trimethylsilyl)ethyl (TMSE) ester using standard conditions, followed by deprotection of the three protected hydroxy groups to provide derivative [1a]. Treatment of [1a] with 9-fluorenylmethyl 4-(formyl)phenoxyacetate dimethyl acetal [2a] and a catalytic amount of p-toluenesulfonic acid (TsOH) gave the 9-fluorenylmethyl (Fm)-protected benzylidene acetal [3a]. Following several protecting-group manipulation steps (protecting the anomeric OH with allyloxycarbonyl group, and removing the Fm carboxylic acid protecting group), the resultant acid was coupled to the aminomethyl-polystyrene resin using EDC/HOBt conditions. The loading was assessed qualitatively using the Kaiser test for free amine residues and gravimetrically by releasing the product off the resin with 90% aqueous acetic acid (AcOH) containing 1% trifluoroacetic acid (TFA). Released product showed no detectable cleavage of the acid labile anomeric allyloxycarbonyl (Alloc) group.

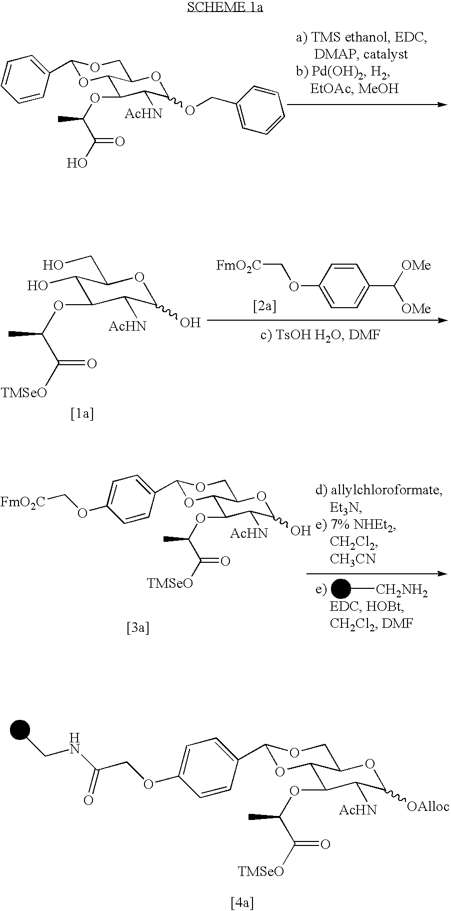

### EXAMPLE 2

### Synthesis of Resin-Bound N-Acetylmuramic Acid—Attachment of MurNAc to Resin-Bound Linker

An alternative method for the preparation of resin-bound N-acetylmuramic acid [4a] is depicted in Scheme 2a, and described in detail herein below.

Synthesis of [5]

To a vessel containing 600 mg of aminomethyl, uniform bead polystyrene resin (1.1 mmol/g loading) was added in 15 ml of wet dimethyl formamide (DMF), followed by 0.89 g of 1-hydroxybenzotriazole (HOBt), 1.27 g of 1-ethyl-3-(3-dimethylaminopropyl)carbodiimide (EDC), 506 μL of triethylamine (TEA), and 595 mg of 4-formylphenoxyacetic acid. The vessel was shaken for 30 minutes then left sitting at ambient temperature overnight. The solvent was drained and the resin washed extensively with DMF, methanol, water, and dichloromethane (DCM) and dried under vacuum to yield 730 mg of dry resin.

Synthesis of [6a]

Dry resin from the previous step was swelled in 10 mL of dry DCM and treated with 430 μL of trimethylorthoformate (TMOF) and catalytic p-toluenesulfonic acid (TsOH) at ambient temperature overnight. The resin was washed with anhydrous DCM and dried under vacuum to yield 1.38 g of resin.

Synthesis of [7a]

To 200 mg of resin [6a] swelled in 5 mL of DMF, 118 mg of diol [1a] and a catalytic amount of TsOH were added. The mixture was left at ambient temperature overnight then washed with DMF, methanol and DCM and dried to yield 175 mg of dried resin.

Synthesis of [4a]

To 80 mg of resin [7a] swelled in 1 mL of DCM, 16 μL of TEA, 9 μL of allyloxychloroformate, and catalytic amount of 4-N, N-dimethylaminopyridine (DMAP) was added. The reaction vessel was shaken at ambient temperature for 2 hours then washed extensively with DCM and DMF and dried under vacuum.

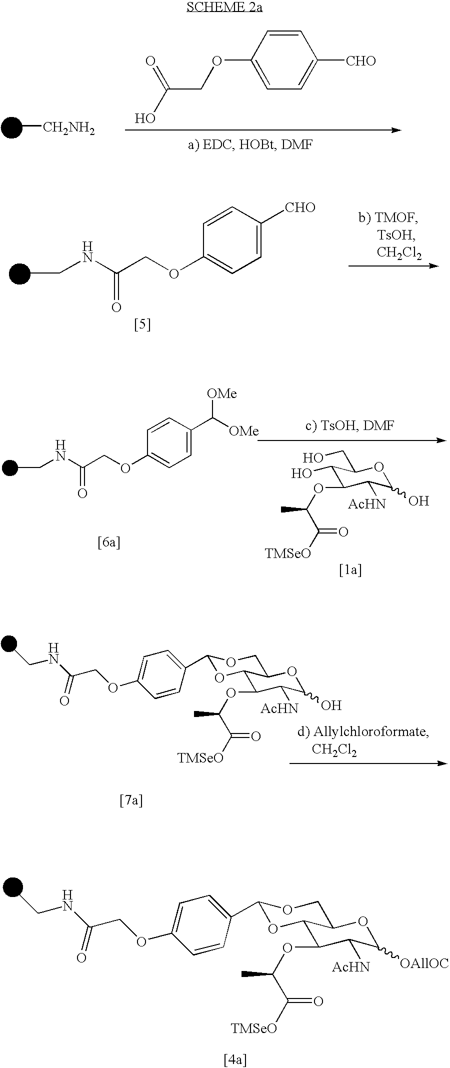

### EXAMPLE 3

### Synthesis of N-Acetylmuramic Acid Derivatives

The procedure for the preparation of N-acetylmuramic acid derivatives is depicted in Scheme 3a, and described in detail herein below.

Synthesis of [8a]

To a vessel containing 470 mg of resin [4a] swelled in 5 mL of dry tetrahydrofuran (THF), 0.88 mL of 1.0 M solution of tetrabutylammonium fluoride in THF was added. The reaction mixture was shaken at ambient temperature for 2 hours. The resin was washed extensively with DMF, then dried to yield 487 mg of resin. The above-described resin was added to three separate vessels (28 mg in each vessel) and swelled in 0.6 mL of DMF. Then 6.5 mg of HOBt and 9.2 mg of EDC were added to each vessel. Finally one of the following three acids was added to each vessel: β-alanine, L-serine, and D-glutamate. The reaction vessels were shaken for 3 hours, then washed dried and washed with DMF and DCM and combined in a single tube to yield 78 mg of material.

Synthesis of [9a]

Resin [8a] (75 mg) was swelled in 0.5 mL of dry DCM. To the mixture 0.12 mL of phenylsilane and 5.5 mg of palladium tetrakis-triphenylphospine were added. The reaction vessel was rotated at ambient temperature for 2 hours. Then washed with DMF and DCM and dried.

The above resin was split into 3 tubes each containing 22 mg of material and swelled in 0.5 mL of DMF. To each vessel was added one of the following reagents: p-chlorobenzenesulfonylisocyanate (12 μL), o-toluenesulfonylisocyanate (12 μL), or benzenesulfonylisocyanate (11 μL). The reactions were rotated overnight, the washed extensively with methanol, DMF, and DCM and dried.

Synthesis of Ia′

Resin [9a] were mixed in a single vessel and swelled in THF then treated with 1 M TBAF in TBF (0.23 mL) for 3 hours. The resin washed extensively with DMF, THF, methanol and DCM and dried. Approximately 25 beads were picked, placed into an HPLC vial and treated with 15% TFA in DCM containing 1% water for 15 minutes. The cleavage solution was then evaporated and 20 μL of acetonitrile were added. The acetonitrile solution was analyzed by LC/MS (ESI mass spectrometry). All nine compounds were present as judged by the HPLC trace and the MS detector.

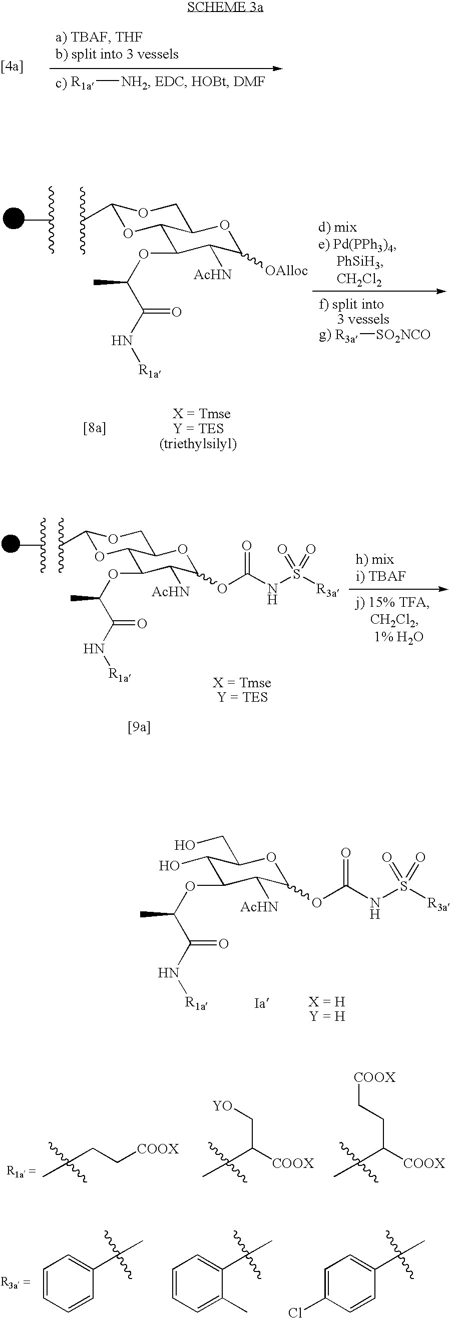

### EXAMPLE 4

### Synthesis of N-acetylmuramic Acid Derivatives

Compounds [1b-e] were prepared in accordance with the procedure previously described in Scheme 4. Compounds [1a-e] were attached to resin [6a] (20 mg each in 5 separate vessels) following procedures for preparation of [7a] previously described in Example 2. Resin in each vessel was then swelled in 0.5 mL of dry DCM and treated with 27 μL of allylchloroformate and 55 μL of triethylamine and 1 mg of DMAP at 0° C. for 30 minutes then ambient temperature for 3 hours. The resin was extensively washed with DMF and DCM. To each vessel was added 0.5 mL of THF, then 60 μL of TBAF in THF and rotated for 2 hours. After extensive washing with THF and DMF and drying, the resins were suspended in 1 mL of DMF. To each tube was added a cocktail containing 17 mg of L-glutamic acid dimethyl ester, 15 mg of EDC, 11 mg of HOBt and 15 μL of TEA. The five tubes with resin were shaken at ambient temperature overnight, then washed extensively as before. Each tube was treated with a solution of 20% piperidine in DMF, 0.25 mL, for 1.5 hours, then the solution was drained and the resin washed and dried as described earlier. Resins in vessels a-e were swelled in DMF and then treated with following reagents at ambient temperature overnight:

Vessel a: 20 mg 2,4-dichlorocinnamic acid, 26 mg of EDC and catalytic amount of DMAP.

Vessel b: 40 mg N-methyl-N-phenylcarbamoyl chloride and 45 μL of TEA

Vessel c: 14 μL benzenesulfonylisocyanate

Vessel d: 16 μL p-chlorophenylsulfonylisocyanate

Vessel e: 20 mg 4-oxy-4-(3-thienyl)-butyric acid, 26 mg of EDC and catalytic amount of DMAP.

Each vessel was drained and extensively washed with methanol, DMF and DCM and dried under vacuum. Approximately 20 beads were collected from each vessel and treated with 15% TFA in DCM containing 1% water for 15 minutes. The cleavage solution was then evaporated and 20 μL of acetonitrile were added. The acetonitrile solution was analyzed by LC/MS (ESI mass spectrometry). The masses of all the desired compounds [Ia a-e] were found in the HPLC/ MS trace. The N-acylmuramic acid derivatives obtained are shown in Table 1.

TABLE I

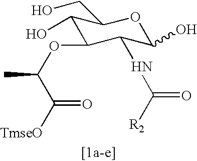

| [1] | R₂ |
|---|---|
| a | methyl |
| b | ethyl |
| c | cyclopropyl |
| d | methoxymethyl |
| e | n-butylamino |

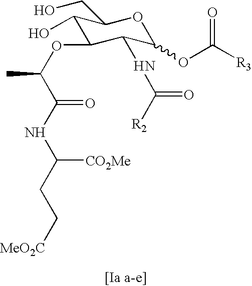

| Ia | R₂ | R₃ |
|---|---|---|
| a | methyl | 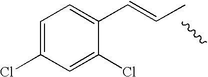 |
| b | ethyl | 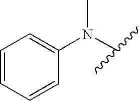 |
| c | cyclopropyl | 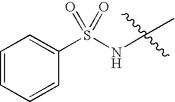 |
| d | —CH₂OCH₃ | 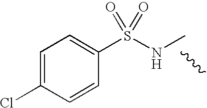 |
| e | n-butylamino | 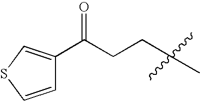 |

What is claimed is:

1. A compound of formula Ia: 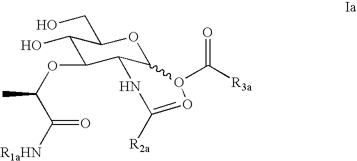
or a pharmaceutically acceptable salt thereof wherein
R₁ₐ is selected from:
1) —C₁₋₁₀alkyl-CO₂R^{a},
2) —CH(CO₂R^{a})CH₂CH₂CO₂R^{b}, and
3) —CH(CO₂R^{a})CH₂OR^{b};
R₂ₐ is selected from:
1) C₁₋₁₀ alkyl,
2) —NR^{a} (C ₁₋₁₀ alkyl),
3) —CH₂OR^{a},
4) C₃₋₆cycloalkyl,
5) Ar, and
6) —N(R^{a})—Ar;
R₃ₐ is selected from:
1) —N(R^{a})—Ar,
2) —CH═CH₂—Ar,
3) —NHSO₂—Ar, and
4) —(CH₂)₂₋₅—C(O)—3-thienyl;
Ar is phenyl optionally substituted with 1 to 2 groups independently selected from halogen and C₁₋₄alkyl; and
R^{a} and R^{b} are independently selected from hydrogen and C₁₋₁₀ alkyl.

2. A method for preparing a resin-bound compound of Formula [7]: 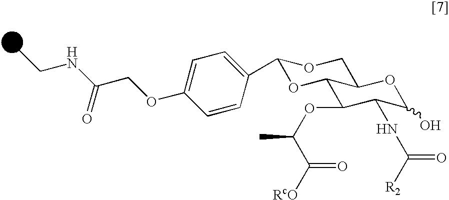
wherein
● represents a polymeric resin support, and R^{c} is a carboxy protecting group, which comprises:
coupling a monosaccharide of Formula [1]: 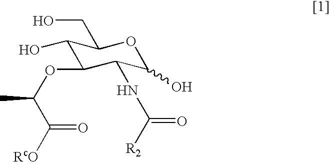
to an activated polymeric resin support of Formula [6]: 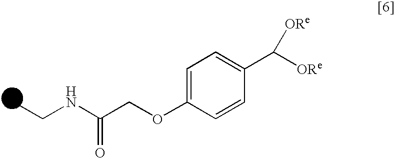
wherein R^{e} is C₁₋₃alkyl.

3. A method of claim 2, which further comprises:
(a) coupling 2-(4-formylphenoxy)acetic acid to a polymeric resin support having a free amino group; and
(b) activating the resin-bound 2-(4-formylphenoxy) acetate as an acetal to provide the activated polymeric resin support of Formula [6].

4. The method of claim 3 wherein the polymeric resin support is aminomethyl polystyrene uniform beads.

5. A method for preparing a resin-bound compound of Formula [4] 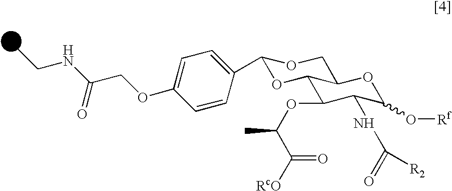
which comprises:
(a) coupling the monosaccharide of Formula III to carboxy protected 4-(formyl)phenoxyacetic acid di(C₁₋₃alkyl) acetal to form an intermediate of Formula [3] 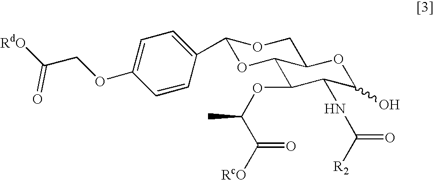
wherein R^{c} and R^{d} are different carboxy protecting groups, and Rf is a hydroxy protecting group;
(b) introducing the Rf hydroxy protecting group;
(c) removing the carboxy protecting group R^{d}; and
(d) coupling the deprotected compound of Formula IIa to a resin having free amino group to provide the resin-bound compound of Formula [4].

6. The method of claim 5 wherein the polymeric resin support is aminomethyl polystyrene uniform beads.

7. A method for preparing a library of compounds of Formula I 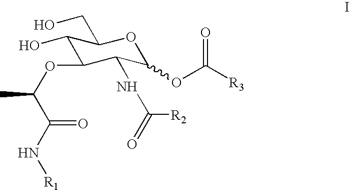
wherein R₁, R₂ and R₃ are independently an organic radical, which comprises:
a) removing one of the protecting groups R^{c} or R^{f} from a compound of formula [4] 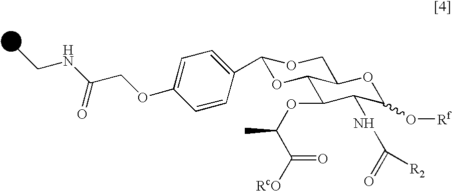
wherein ● represents a polymeric resin support, R^{c} is a carboxy protecting group and Rf is a hydroxy protecting group, to provide a first functional group,
b) derivatizing said first functional group,
c) removing the second protecting group from the compound of formula [4] to provide a second functional group,
d) derivatizing said second functional group, and
e) releasing modified compounds of formula I from the resin.

8. A library of compounds prepared by the method of claim 7 for screening for inhibiting Mur enzymes.

9. A pharmaceutical composition which is comprised of a compound in accordance with claim 1 in combination with a carrier.

10. A method of treating a bacterial infection in a mammalian patient in need of such treatment which is comprised of administering to said patient a compound in accordance with claim 1 in an amount which is effective for treating a bacterial infection.
